(19) United States
(12) Patent Application Publication
(10) Pub. No.: US 2005/0163668 A1
(43) Pub. Date: Jul. 28, 2005

(54) Disinfecting system

(76) Inventors: Keith M. Crawford, Valencia, CA (US); Richard N. Metzger, Valencia, CA (US); Michael A. Pedersen, Valencia, CA (US)

Correspondence Address: CISLO & THOMAS, LLP, 233 WILSHIRE BLVD, SUITE 900, SANTA MONICA, CA, 90401-1211, US

(21) Appl. No.: 10/969,162

(22) Filed: Oct. 19, 2004

Related U.S. Application Data
(60) Provisional application No. 60/535,291, filed on Jan. 9, 2004.

(51) Int. Cl. A61L 9/20

(52) U.S. Cl. 422/121; 422/24

(57) ABSTRACT

A disinfecting system comprises a UVC (Ultraviolet C) irradiating device adapted for use as a disinfectant in fluid conveying equipment, and a support assembly adapted to control the operation of the UVC irradiating device. The support assembly is removably mounted to the fluid conveying equipment via a mounting ring. The mounting ring incorporates a safety actuator. The support assembly includes an electrical interlock switch adapted to cooperate with the safety actuator to prevent the unsafe operation of the UVC device within the fluid conveying equipment based on disposition of the safety actuator within the mounting ring.

### CROSS-REFERENCE TO RELATED APPLICATION

This application claims priority to U.S. Provisional Application No. 60/535,291, filed on Jan. 9, 2004, the entire contents of which are hereby incorporated by reference.

### BACKGROUND

Electromagnetic radiation including ultraviolet (UV) energy in the C-band may be used for the disinfection of surfaces and fluids, including airstreams. Specifically, UVC energy has been used in HVAC (Heating, Ventilating and Air Conditioning) systems to disinfect airstreams and prevent microbial growth therein.

A UVC lamp may be mounted within, or on, a robust steel or plastic enclosure. The enclosure may contain the necessary electrical components to deliver the appropriate power to the UVC lamp. Often the entire appliance may be unwieldy, awkward and expensive. In addition, installation of the equipment may be difficult. The fixture may be installed within the HVAC system, or on the exterior surface of the HVAC system. When installed within the HVAC system, a suitable support structure may be needed to support the UVC fixture as well as an electrical junction box, which may be used to provide power to the fixture. When the fixture is installed on an exterior surface of the HVAC system, it may consist of an enclosure with the UVC lamp protruding within the HVAC system. The enclosure may take a significant amount of space, and may also require additional hardware for supplying power to the fixture.

Radiation emitted from UVC lamps can be detrimental to the physical health of directly exposed users or maintenance personnel. It is be important that safety devices be provided to prohibit users or maintenance personnel from being exposed to, and irradiated by, UVC light.

### SUMMARY

Exemplary embodiments disclosed herein are directed to a disinfecting system.

In accordance with one aspect of the invention, a disinfecting system comprises at least one irradiating device adapted for use as a disinfectant in fluid conveying equipment, and at least one support assembly adapted to control the operation of the irradiating device. The disinfecting system also comprises means for safely operating the irradiating device within the fluid conveying equipment for fluid disinfection when the irradiating device is operatively coupled to the support assembly. The support assembly is removably mounted to the fluid conveying equipment.

In accordance with another aspect of the invention, a modular disinfecting system comprises a mounting portion configured to couple to a system, and a modular housing configured to couple to the mounting portion. The modular housing is configured to receive a selectively couplable electromagnetic energy source, to house control electronics and power connections for said modular system, and includes a safety actuator. The electromagnetic energy source is activated when the safety actuator is activated.

In accordance with yet another aspect of the invention, a modular device for disinfecting air, fluid or surfaces in HVAC equipment, duct or other system comprises a mounting portion configured to couple to a system, a modular housing including a safety actuator, configured to couple to the mounting portion, and an electromagnetic radiation source configured to couple to the modular housing. The safety actuator is activated when the housing is rotated into an actuated position. The electromagnetic energy source is activated when the safety actuator is activated.

In accordance with still another aspect of the invention, a device for disinfecting air or surfaces in a system comprises a mounting portion configured to couple to a system, and a modular housing configured to couple to the mounting portion. The modular housing comprises an electromagnetic radiation source, electronics to control the operation of the electromagnetic radiation source, a safety actuator configured to actuate, and allow power to the electromagnetic radiation source when the modular housing is in an actuated position, and an indicator to indicate proper operation of the electromagnetic energy source. The safety actuator is actuated when the modular housing is twist-coupled to the mounting portion, thereby causing the safety actuator to be moved into an actuated position.

These and other aspects of the invention will become apparent from a review of the accompanying drawings and the following detailed description of the invention.

### BRIEF DESCRIPTION OF THE DRAWINGS

The invention is generally shown by way of reference to the accompanying drawings in which:

FIG. 1 is a cross section of an exemplary embodiment of a device installed in HVAC duct.

FIG. 2 is a detailed view of components in a handle and mounting ring, according to an exemplary embodiment.

FIG. 3 is a detailed view of lamp base in a handle of the device, according to an exemplary embodiment, showing how the lamp is retained.

FIG. 4 is an isometric view of the handle showing the location of a safety switch actuator, according to an exemplary embodiment.

FIG. 5 is a detailed view showing an actuator and safety switch, according to an exemplary embodiment.

### DETAILED DESCRIPTION

The detailed description set forth below in connection with the appended drawings is intended as a description of exemplary embodiments and is not intended to represent the only forms in which the exemplary embodiments may be constructed and/or utilized. The description sets forth the functions and the sequence of steps for constructing and operating the exemplary embodiments in connection with the illustrated embodiments. However, it is to be understood that the same or equivalent functions and sequences may be accomplished by different embodiments that are also intended to be encompassed within the spirit and scope of the invention.

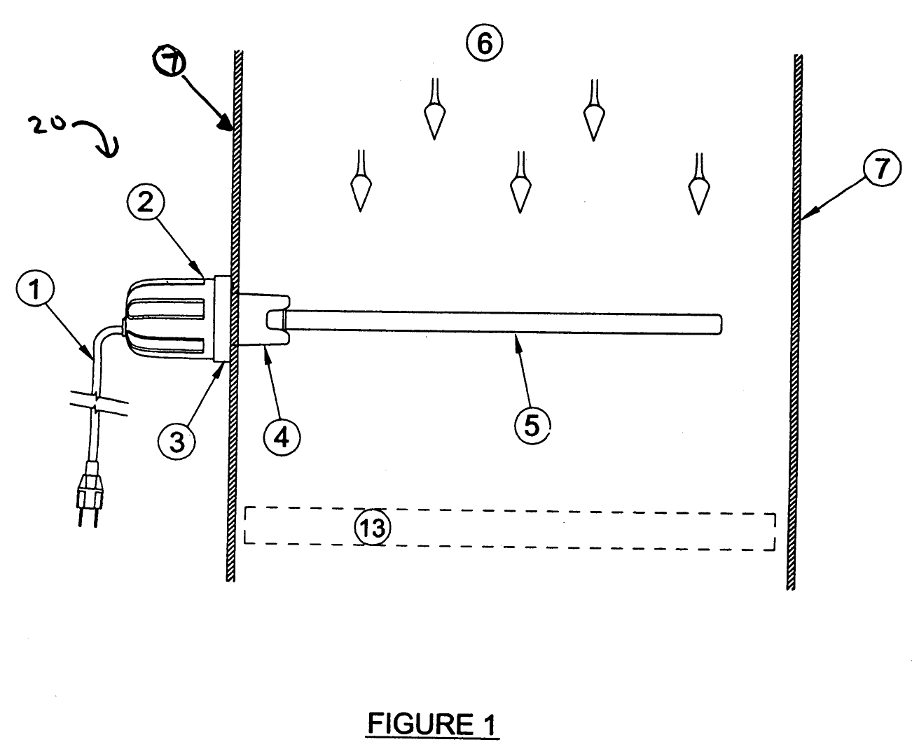

Some embodiments of the invention will be described in detail with reference to the related drawings of FIGS. 1-5. Additional embodiments, features and/or advantages of the invention will become apparent from the ensuing description or may be learned by practicing the invention. In the figures, the drawings are not to scale with like numerals referring to like features throughout both the drawings and the description.

The present invention is generally directed to a low profile, easy to use integrated lamp fixture, which requires very little space on the exterior of an HVAC system. Embodiments may be comprised of user-friendly, safe, UV irradiating devices of minimal size and complexity for use in HVAC (Heating, Ventilating and Air Conditioning) equipment, or air ducts, and the like.

The integrated lamp fixture may incorporate two main components into a safe and effective UV product. The first component may be a lamp assembly that integrates a UV lamp, electrical enclosure, electronics, mounting features electrical connection and user visual indicators into one enclosure comprising of two plastic enclosure components.

The second component may be a mounting ring, which mounts to the HVAC equipment or duct and may provide a quick, robust configuration to affix the removable lamp assembly with a partial turn of a knob. The lamp handle may include a line cord to provide electrical power to the lamp, and the mounting ring may include an actuator to close an electric safety switch.

All electronics for power and control may be incorporated within the mounting handle. A double acting safety interlock switch may also be incorporated. An actuator may be incorporated in the mounting ring, and a switch may be included in the handle assembly. This may require the lamp to be in a proper position, i.e. the unit to be mounted properly before power is supplied to the lamp.

The device may be cost effective to produce, require very little space to mount, and be easy to install and use while still maintaining the safety of users and/or maintenance personnel.

FIG. 1 shows a cross section of an exemplary embodiment of a disinfecting system 20 installed on a typical HVAC duct or other system. The modular housing assembly 2 contains all of the electronics for powering the lamp and control of the lamp. A line cord 1 is included in a handle portion of modular housing assembly 2 allowing typical household voltages and currents, or other voltages and currents, to be utilized for powering the lamp assembly 2 and system. A disinfecting system 20 may be mounted to the HVAC equipment or other system 7 via a mounting ring 3.

Mounting ring 3 may be easily affixed to HVAC equipment or other system 7, and a hole or other configuration in the system may allow the electromagnetic radiation source to protrude into the system or adjacent the material to be disinfected. The electromagnetic radiation source may be installed perpendicular to the airflow 6, however, it will be appreciated that other configurations may be utilized, as desired. In an HVAC system and other systems, UVC may be utilized to irradiate filters or air conditioning coil 13 or fluids or other surfaces, as desired. A lamp base 4 may also be included in the modular housing assembly. Lamp base 4 may be configured to couple to an electromagnetic radiation source 5. Lamp base 4 may incorporate a snap portion, or be keyed to insure use of the proper type or style of UV lamp. Furthermore, other coupling configurations and electromagnetic radiation sources may be utilized, as desired.

Modular housing 2 may be made of plastic, metal, polymers, or other materials, or combinations thereof, as desired. Mounting ring 3 may be made of similar materials, and may be configured to fit into an orifice formed in a system to mount to it. However, other coupling configurations may be utilized to affix mounting ring 3 to a system.

The modular design of the system may make it easier for a user to remove the electromagnetic radiation source 5 from the system and replace it easily. Lamp base 4 may also be made out of plastic, metal, polymers, or other materials and combinations thereof, as desired. Electromagnetic radiation source 5 may be an ultraviolet light source, however, other electromagnetic radiation sources may be utilized to disinfect systems, surfaces, fluid, air, or other items, as desired. System 7 may be an HVAC, duct, or other system that may utilize an electromagnetic radiation source to disinfect air, fluid, surfaces, filters, air conditioning coils, and other devices and surfaces, as desired.

It will be appreciated that both the disinfecting system 20 and system 7 may be of many sizes and configurations to utilize system 20 as a disinfecting source.

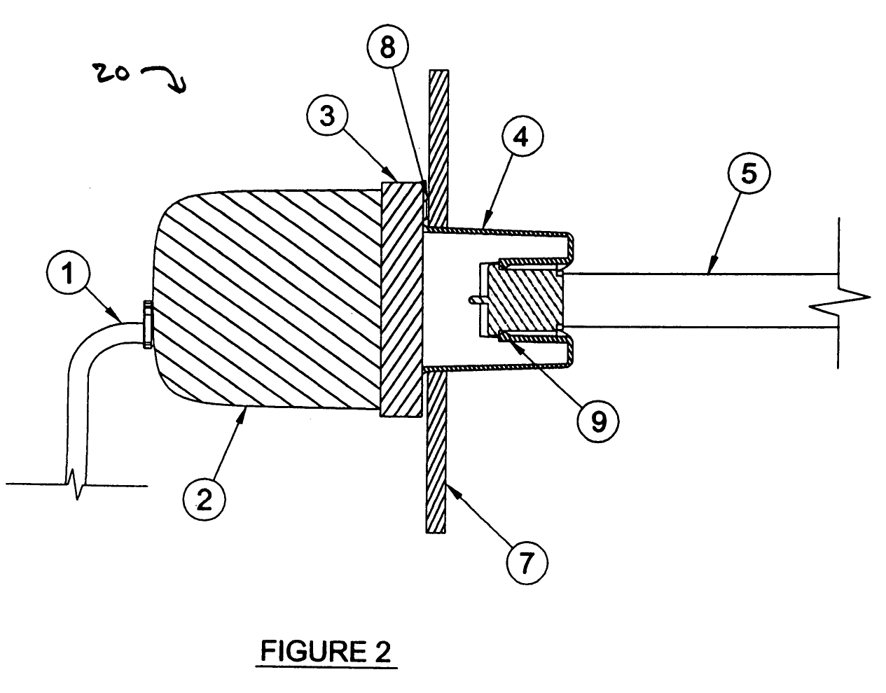

FIG. 2 shows a more detailed cross section of the assembly of the embodiment of FIG. 1. In addition to the features previously described, a more detailed view of the lamp base 4 may be seen. The electromagnetic radiation source 5 may be a standard commercially available UVC type lamp, or other electromagnetic radiation source, or other disinfecting device, which may be customized to snap fit into a coupling structure 9, which may be incorporated within the lamp base 4. A safety actuator 8 may be incorporated into the mounting ring 3, or the modular housing 2, or other configuration. Furthermore, safety actuator 8 may be held into its proper, or actuated, position by the HVAC duct 7 when the mounting ring 3 is installed adjacent the duct 7.

Safety actuator 8 may be configured so that power to the electromagnetic radiation source 5 will be disconnected when a user attempts to replace the electromagnetic radiation source. With this configuration, a user may not be exposed to electromagnetic radiation, UV light, or other harmful effects of the system, as the electromagnetic radiation source will be powered down when not in an operative position with respect to system 7, or other system.

Furthermore, the lamp base 4 may include a coupling structure 9 that may be configured to couple to electromagnetic radiation source 5 via the electromagnetic radiation source coupling structure, such that the electromagnetic radiation source 5 will not inadvertently uncouple from lamp base 4 during the installation of the system, during use, or any other time when it is not desired.

Again, system 20 includes a modular housing 2, line cord 1, and mounting ring 3. Lamp base 4 may be configured to couple to commercially available UV sources and lamps, or may be configured in another manner to allow the electromagnetic source 5 to selectively and securely couple to the system.

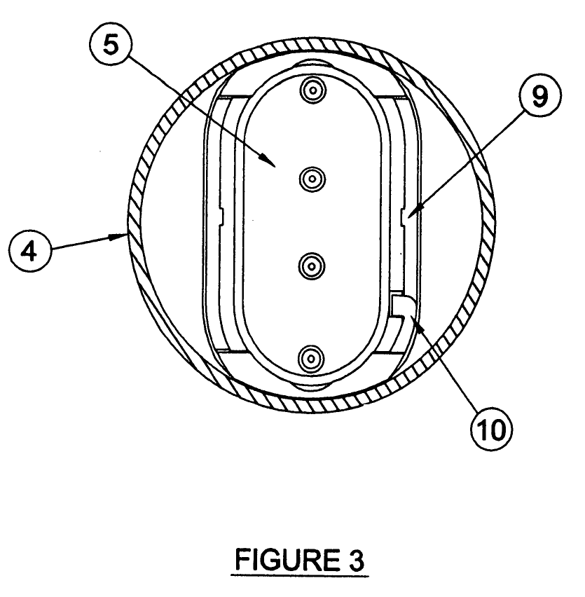

FIG. 3 shows the detail of the cross section of the lamp base 4, looking axially from the direction of the electromagnetic radiation source, according to an exemplary embodiment. In FIG. 3, the coupling structure 9, and an electromagnetic radiation source coupling structure 10, may be seen in more detail.

FIG. 3 may better show a configuration for lamp base 4, electromagnetic radiation source 5, and how they may couple together via coupling structure 9 and electromagnetic radiation coupling structure 10 such that the electromagnetic radiation source 5 and lamp base 4 will not inadvertently uncouple when not desired. It will be appreciated that other coupling configurations and structures may be utilized to couple lamp base 4 and electromagnetic radiation source 5, as desired.

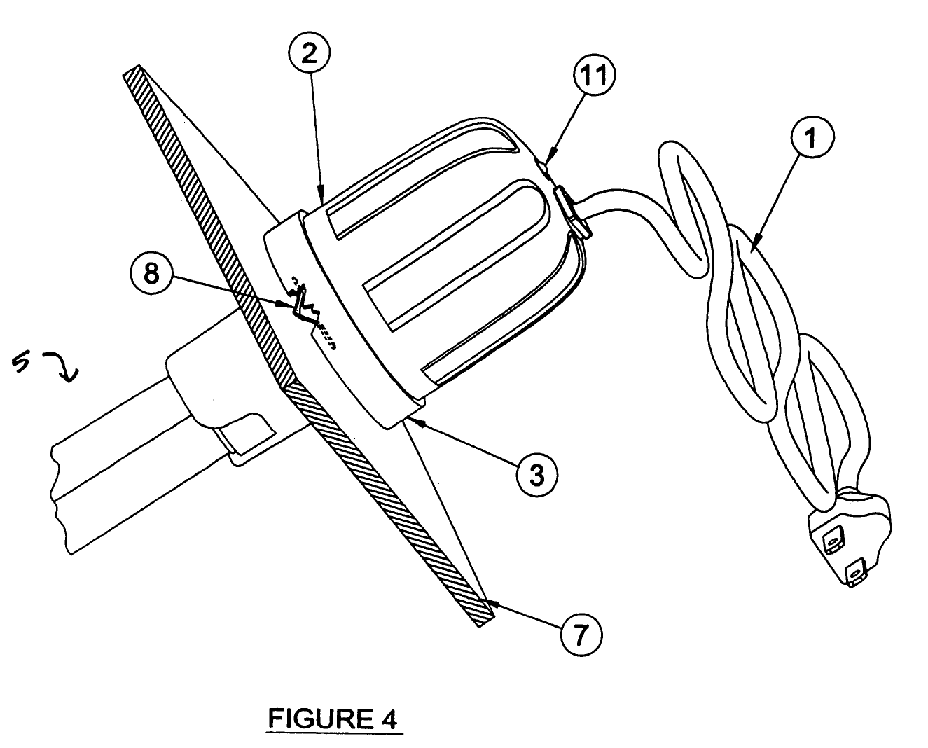

FIG. 4 shows an isometric view of the integrated lamp assembly 2 according to an exemplary embodiment. In addition to similar features previously described, an indicator 11 may be added to indicate proper operation of the electromagnetic radiation source. A safety actuator 8 is shown in its proper position when the mounting ring 3 is installed adjacent the HVAC duct system 7.

Safety actuator 8 may be made of metal, plastic polymers, or combinations thereof, or other materials as desired. Safety actuator 8 may then mechanically actuate to an electronic switch or other means of allowing power to pass to the electromagnetic radiation source 5, such that the electromagnetic radiation source 5 will not be powered unless the system is in an actuated position or in a safe position, as desired. Indicator 11 may be a light emitting diode (LED), however other indicators may be utilized, as desired. Indicator 11 may indicate that the system is operating properly, that the electromagnetic source 5 is operating properly, or combinations thereof to indicate different operation modes and situations, as desired. Although one indicator is shown, it will be appreciated that any number of indicators may be utilized, as desired.

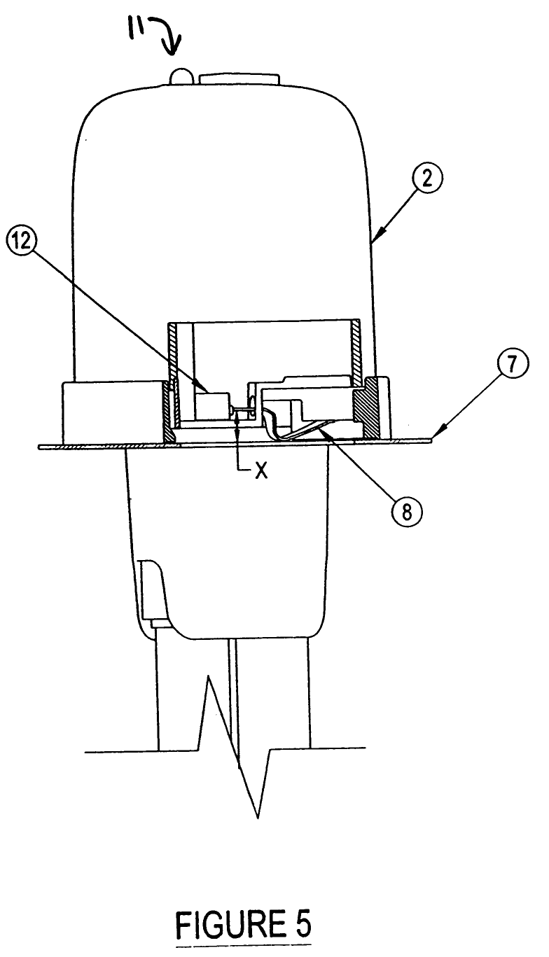

FIG. 5 may show the details of the components of the safety actuator feature, according to an exemplary embodiment. An electrical interlock switch 12 may be incorporated into the handle assembly 2. A metal or plastic safety actuator may be incorporated into the mounting ring 3. When the mounting ring 3 is installed properly to the HVAC duct 7, the safety actuator 8 may be positioned into its proper location with a dimension X. When the handle incorporating the lamp is placed into the mounting ring 3 and rotated into an actuated position, the electrical interlock switch 12 may be actuated by the safety actuator 8 if dimension X is correct.

Electric interlock switch 12 may be a solid-state relay, or other switch that may be utilized to indicate that safety actuator 8 is in a safe position. Interlock switch 12 may also be a switch that indicates to the overall system that it is safe to power the electromagnetic radiation source, or other configuration or indication, as desired.

Indicator 11 may extend away from modular housing 2 however it may be contained within modular housing 2 to insure that it would not get broken or damaged during an installation, or when a user changes electromagnetic radiation sources, or at other times.

The overall system may be configured such that distance X may be a small or large distance, as desired. Furthermore, other configurations of the system and of the configuration of the elements may be utilized, as desired.

In closing, it is to be understood that the exemplary embodiments described herein are illustrative of the principles of the present invention. Other modifications that may be employed are within the scope of the invention. Thus, by way of example, but not of limitation, alternative configurations may be utilized in accordance with the teachings herein. Accordingly, the drawings and description are illustrative and not meant to be a limitation thereof. Moreover, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Thus, it is intended that the invention cover all embodiments and variations thereof as long as such embodiments and variations come within the scope of the appended claims and their equivalents.

1. A disinfecting system, comprising:
at least one irradiating device adapted for use as a disinfectant in fluid conveying equipment;
at least one support assembly adapted to control the operation of said at least one irradiating device; and
means for safely operating said at least one irradiating device within the fluid conveying equipment for fluid disinfection when said at least one irradiating device is operatively coupled to said at least one support assembly, said at least one support assembly being removably mounted to the fluid conveying equipment.

2. The disinfecting system of claim 1, further comprising means for operatively coupling said at least one irradiating device to said at least one support assembly.

3. The disinfecting system of claim 1, further comprising means for removably mounting said at least one support assembly to the fluid conveying equipment.

4. The disinfecting system of claim 3, wherein said removable mounting means includes at least one fluid conveying equipment-mounting ring.

5. The disinfecting system of claim 4, wherein said safely operating means includes at least one electrical interlock switch being incorporated into said at least one support assembly and at least one safety actuator being incorporated into said at least one fluid conveying equipment-mounting ring,

6. The disinfecting system of claim 5, wherein said at least one electrical interlock switch cooperates with said at least one safety actuator to prevent the unsafe operation of said at least one irradiating device within the fluid conveying equipment based on disposition of said at least one safety actuator within said at least one fluid conveying equipment-mounting ring.

7. The disinfecting system of claim 1, wherein said at least one irradiating device is adapted for use as a fluid disinfectant in HVAC (Heating, Ventilation, and Air Conditioning) equipment.

8. The disinfecting system of claim 7, wherein said at least one irradiating device operates as a UVC (Ultraviolet C) energy source.

9. The disinfecting system of claim 1, wherein said at least one support assembly comprises means for indicating the proper operation of said at least one irradiating device when said at least one irradiating device is operatively coupled to said at least one support assembly within the fluid conveying equipment.

10. The disinfecting system of claim 9, wherein said indicating means includes at least one LED (Light Emitting Diode) being operatively coupled between said at least one irradiating device and at least one power supply.

11. The disinfecting system of claim 2, wherein said coupling means includes a base portion adapted to removably couple said at least one irradiating device to said at least one support assembly.

12. The disinfecting system of claim 11, wherein said base portion is an integral part of said at least one support assembly.

13. A modular disinfecting system, comprising:
a mounting portion configured to couple to a system; and
a modular housing configured to couple to said mounting portion;
wherein said modular housing is configured to receive a selectively couplable electromagnetic energy source, to house control electronics and power connections for said modular system, and includes a safety actuator, and
wherein said electromagnetic energy source is activated when said safety actuator is activated.

14. The modular disinfecting system of claim 13, further comprising a safety interlock configured to power said electromagnetic energy source when said system is in an operative position.

15. The modular disinfecting system of claim 13, further comprising an indicator that indicates when said electromagnetic energy source is operating properly.

16. The modular disinfecting system of claim 13 wherein said safety actuator is actuated when said modular housing is twist-coupled to said mounting portion, thereby causing said safety actuator to be moved into an actuated position.

17. A modular device for disinfecting air, fluid or surfaces in HVAC equipment, duct or other system, comprising:
a mounting portion configured to couple to a system;
a modular housing including a safety actuator, configured to couple to said mounting portion; and
an electromagnetic radiation source configured to couple to said modular housing,
wherein said safety actuator is activated when said housing is rotated into an actuated position, and
wherein said electromagnetic energy source is activated when said safety actuator is activated.

18. The modular device of claim 17, wherein said modular housing further houses electronics to control said system.

19. The modular device of claim 17, further comprising an indicator that indicates when said electromagnetic energy source is operating properly.

20. The modular device of claim 17, wherein said electromagnetic energy source is selectively couplable to said modular housing.

21. The modular device of claim 17, wherein said safety actuator is actuated when said modular housing is twist-coupled to said mounting portion, thereby causing said safety actuator to be moved into an actuated position.

22. A device for disinfecting air or surfaces in a system comprising:
a mounting portion configured to couple to a system; and
a modular housing configured to couple to said mounting portion,
wherein said modular housing comprises:
an electromagnetic radiation source;
electronics to control the operation of said electromagnetic radiation source;
a safety actuator configured to actuate, and allow power to said electromagnetic radiation source, when said modular housing is in an actuated position; and
an indicator to indicate proper operation of the electromagnetic energy source,
wherein said safety actuator is actuated when said modular housing is twist-coupled to said mounting portion, thereby causing said safety actuator to be moved into an actuated position.

23. The device of claim 22, wherein said electromagnetic energy source is selectively couplable to said modular housing.
